(19) United States
(12) Patent Application Publication
(10) Pub. No.: US 2007/0219468 A1
(43) Pub. Date: Sep. 20, 2007

(54) MONITORING AND TRACKING OF IMPULSES EXPERIENCED BY PATIENTS DURING TRANSPORT

(75) Inventors: Shetal Shah, Great Neck, NY (US); Martha Caprio, North Flushing, NY (US); Allen Mincer, New York, NY (US)

Correspondence Address: DARBY & DARBY P.C., P.O. BOX 770, Church Street Station, New York, NY, 10008-0770, US

(73) Assignee: New York University, New York, NY (US)

(21) Appl. No.: 11/539,909

(22) Filed: Oct. 10, 2006

Related U.S. Application Data
(60) Provisional application No. 60/724,738, filed on Oct. 7, 2005.

(51) Int. Cl. A61B 5/103

(52) U.S. Cl. 600/587; 600/595

(57) ABSTRACT

A detection and monitoring system having an impulse sensor located on or near the skull of the patient. The impulse sensor can be placed on a securing device which can be a band, hat, skullcap, or adhesive to secure the impulse sensor directly to the bandages of the patient. A three-axis accelerometer and/or a one-axis accelerometer can be used as the impulse sensor. The sensor can be linked to a receiver and the link between the sensor and the receiver can be wired or wireless depending on the nature of the patient. The receiver can be part of a monitor or include a transmitter to relay the sensor data to the monitor at a different location. The monitor can include a processor to analyze the sensor data and a memory to store the sensor data. The sensor data can be transmitted and analyzed in real time.

### CROSS REFERENCE TO RELATED APPLICATIONS

The present application claims priority to U.S. Provisional Patent Application No. 60/724,738 filed Oct. 7, 2005. The entirety of the application is incorporated herein by reference.

### BACKGROUND OF THE INVENTION

1. Field of the Invention

The present invention relates generally to medical transport devices. More specifically, the invention monitors and tracks forces and impulses applied to a patient.

2. Description of the Related Art

Medical emergencies frequently require that patients be transported to hospitals or offices for treatment. In the case of trauma patients, neonates and infants, the transport can be harmful because of the patient's vulnerability. In an effort to protect the patient, hospitals and ambulances frequently have transport systems designed to provide safe transportation environments for the patient. Conventional transport systems contain at least a mattress for adult patients and a mattress with an incubator for neonates and infants. The mattress provides patient comfort and protects the patient from directly contacting the gurney or the floor of the incubator. The incubator is used to ensure a safe environment for the infant patient.

Under smooth traveling conditions these transport systems are sufficient to protect the patient from common hazards. However, rough roads and certain speeds can result in conditions that are beyond those which conventional transport systems can protect against. In many cases the impulses applied to the patients due to bumps in the road are too great for a conventional transport mattress to dampen. Frequently, the impulses are sufficient to allow the patient's head to bump against the gurney or the floor of the incubator. This bumping can cause hospital transfer-related morbidity, including intraventricular hemorrhage. Intraventricular hemorrhaging in infants can also lead to cerebral palsy later in life.

Apparatuses have been designed to dampen the impulses applied to the patient, but there is no way for a caregiver to know the magnitude and the number of impulses that could adversely affect the patient. During transport, both the speed of the transport vehicle and the road condition directly affect the magnitude of the impulses. Currently, there is no way to measure the magnitude of the impulses applied to the patient, and to inform the driver or caregiver that the impulses are reaching a critical point so the driver can slow the vehicle or change the route to a less bumpy road.

### SUMMARY OF THE INVENTION

The invention is a detection and monitoring system having an impulse sensor located on or near the skull of the patient. The impulse sensor can be placed on a securing device which can be a band, hat, skullcap, or adhesive to secure the impulse sensor directly to the bandages of the patient. A three-axis accelerometer can be used as the impulse sensor and can be a “low-g” device, which can detect readings of +/−5 g-forces in each of the three orthogonal axes (X-Y-Z). A one-axis accelerometer can also be used to detect impulses in the Z-axis and may be a “high-g” accelerometer.

The sensor can be linked to a receiver and the link between the sensor and the receiver can be wired or wireless depending on the nature of the patient. The sensor data can be in analog or digital form.

The receiver can be part of a monitor or include a transmitter to relay the sensor data to the monitor at a different location. The monitor can include a processor to analyze the sensor data and a memory to store the sensor data either in raw form or after analysis. In one embodiment, the sensor data is transmitted and analyzed in real time. An alternate embodiment can just store the sensor data for later analysis. The memory can also be used to store any or all of the information outlined below for future review. The processor can assist in other tasks and processes, including and not limited to the embodiments described below. Additionally, multiple processors can be used.

The monitor can include a display to display the analyzed sensor data. A caregiver can view the analyzed data and determine the impulses and forces being applied to the patient. This can assist the caregiver to make corrections to the driving speed of the vehicle transporting the patient or take an alternate route to avoid bumpy road conditions.

In another embodiment, the monitor has preset critical values stored that are the values at which the patient is in danger of injury. The processor can detect that the sensor data is approaching the critical value and activate an alarm to notify the caregiver of the impending danger to the patient. The alarm can be an audio alarm, visual alarm, or both. The alarm can sound a tone and/or the tone can increase as the sensor data approaches the critical value. Further, the alarm can activate the display or an LED to provide at least one of a steady, flashing or incremental indicator as the sensor data approaches the critical value.

The monitor can also include an input device to receive commands to power the system on and off, initiate calibration and test procedures and deactivate the alarm. Additionally, the input device allows a caregiver the option to enter information regarding the patient. The information can be used to retrieve the correct critical value or allow the caregiver to enter a critical value. The additional information regarding the patient can include age, weight, sex, physical condition (e.g. pregnant), and nature of injury. This information can be used to determine the correct critical value and, if necessary, the proper factors of safety to warn the caregiver prior to reaching the critical value.

Additional embodiments can include a Global Positioning System (GPS), either separate from or included with the monitor. The GPS can provide position and velocity information regarding the vehicle. The velocity information can be correlated with the sensor data to determine the safe travel speed of the vehicle to keep the forces applied to the patient below the critical value. Route information can be displayed on the display along with the safe travel speed.

The monitor can also include a transmitter/receiver linked to an external radio source or network (e.g. LAN, WAN or Internet) to receive traffic and road condition information. The traffic and road information can be accessed periodically or accessed in real time to allow for “on-the-fly” changes to the travel route of the vehicle. The traffic and road information can be accessed from a local traffic report, updates provided by other caregivers traveling to the same destination and transportation department databases listing the locations of road surface problems (e.g. open construction sites, road imperfections, raised surfaces, steel plates, and potholes).

As an example, the caregiver can input the destination and receive a potential route from his present location to the destination, e.g. the hospital. The route can be analyzed for fastest travel time, traffic conditions and potential road surface problems and can be changed accordingly. Alternately, or in addition, the system can provide the suggested speed of the vehicle while traveling the route and monitor the speed of the vehicle for compliance.

In another embodiment, a camera can be positioned to monitor the patient. The camera can be a full-motion camera or a still-motion camera that takes images at preset intervals. The image data can be transmitted to the display for the caregiver to monitor the patient if they are in separate sections of the vehicle.

In another embodiment, the type of mattress that the patent is resting on can be inputted via the input device. Different mattresses provide different protection to impulse forces and the monitor can take this into account when analyzing and outputting the data. Alternately, or in addition to the mattress, a supplemental support can also be included. The supplemental support can be an adjustable support, e.g. an air mattress, and can be monitored and controlled by the monitor. For example, a pressure monitor and compressor can be used to monitor the status of the supplemental support. As the sensor data approaches the critical value, the supplemental support can be adjusted to dampen the forces applied to the patient, thus lowering the sensor data to an acceptable level below the critical value (e.g. the mattress can be inflated and deflated as necessary). Additionally, the supplemental support can be used for emergency purposes and only deploy if the sensor data exceeds the critical value or exceeds the critical value by a certain percentage.

The above embodiments are described in light of transporting the patient between two facilities by vehicle. In addition, the present invention can also be used for intra-facility transportation between floors and rooms within the same facility.

### BRIEF DESCRIPTION OF THE DRAWINGS

The above and still further objects, features and advantages of the present invention will become apparent upon consideration of the following detailed description of a specific embodiment thereof, especially when taken in conjunction with the accompanying drawings wherein like reference numerals in the various figures are utilized to designate like components, and wherein:

FIG. 1 is a perspective view of the system of the present invention; and

FIG. 2 is a diagram of the system of the present invention.

### DETAILED DESCRIPTION OF THE EMBODIMENTS

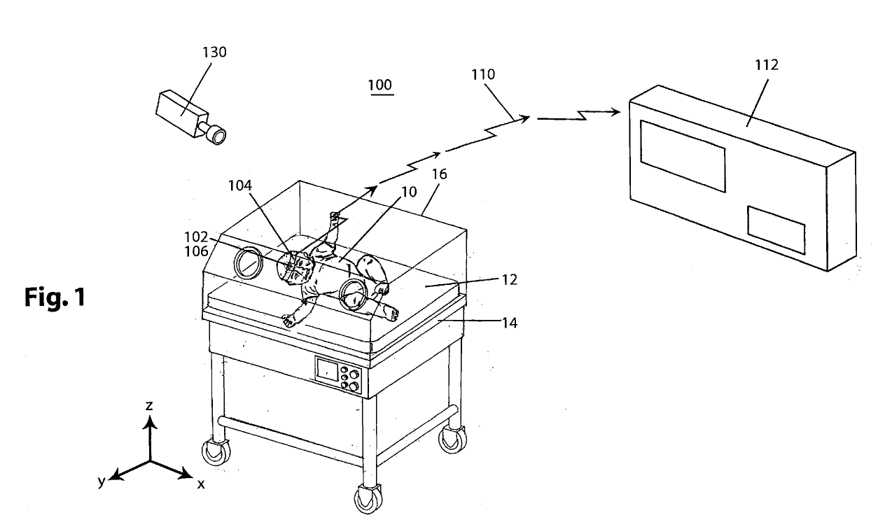

Referring to FIG. 1, a patient 10 is resting on a mattress 12 placed on a gurney 14 to allow for the transportation of the patient 10. The mattress 12 is placed inside an incubator 16 which encloses the patient 10 when the patient 10 is a neonate or infant. Adult trauma patients 10, are placed directly on the mattress 10 and do not require the incubator 16.

The detection and monitoring system 100 of the present invention includes an impulse sensor 102 located on or near the skull of the patient 10. The impulse sensor 102 can be placed on a securing device 104 which can be a band, hat, skullcap, or adhesive to secure the impulse sensor 102 directly to the bandages of the patient 10. A three-axis accelerometer 106 can be used as the impulse sensor 102. The accelerometer 106 can be a “low-g” device, which can detect readings of +/−5 g-forces in each of the three orthogonal axes (X-Y-Z). A one-axis accelerometer 106 can also be used to detect impulses in the Z-axis and may be a “high-g” accelerometer.

The sensor 102 can be linked to a receiver 108 that receives the sensor data 110 from the sensor 102. The link between the sensor 102 and the receiver 108 can be wired or wireless depending on the nature of the patient 10. For example, if the patient 10 is an infant in the incubator 16, the link can be wireless to “pass through” the walls of the incubator 16. The sensor data 110 can be in analog or digital form.

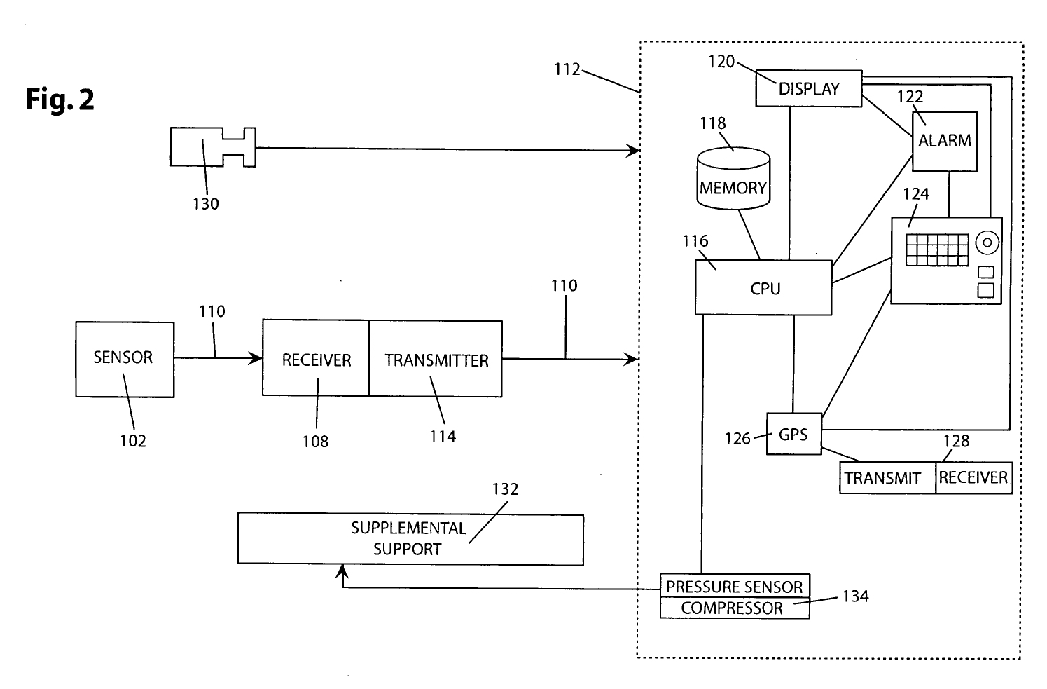

FIG. 2 illustrates that the receiver 108 can be part of a monitor 112, or may include a transmitter 114 to relay the sensor data 110 to the monitor 112 at a different location. The monitor 112 can include a processor 116 to analyze the sensor data 110 and a memory 118 to store the sensor data 110, either in raw form or after analysis. In one embodiment, the sensor data 110 is transmitted and analyzed in real time. An alternate embodiment can just store the sensor data 110 for later analysis. The processor 116 can assist in other tasks and processes, including but not limited to the embodiments described below. Additionally, multiple processors can be used.

The monitor 112 can include a display 120 to display the analyzed sensor data 110. A caregiver can view the analyzed data and determine the impulses and forces being applied to the patient 10. The displayed data 110 can assist the caregiver to make corrections to the driving speed of the vehicle transporting the patient 10 or take an alternate route to avoid bumpy road conditions.

In another embodiment, monitor 112 has preset critical values stored that are the values at which the patient 10 is in danger of injury. The processor 116 can detect that the sensor data 110 is approaching the critical value and activate an alarm 122 to notify the caregiver of the impending danger to the patient 10. The alarm 122 can be an audio alarm, visual alarm, or both. The alarm 122 can sound a tone and/or the tone can increase as the sensor data 110 approaches the critical value. Further, the alarm can activate the display 120 or an LED to provide at least one of a steady, flashing or incremental indicator as the sensor data 110 approaches the critical value.

The monitor 112 can also include an input device 124 to receive commands to power the system 100 on and off, initiate calibration and test procedures and deactivate the alarm 122. Additionally, input device 124 allows a caregiver the option to enter information regarding the patient 10. The information can be used to retrieve the correct critical value or allow the caregiver to enter a critical value.

The additional information regarding the patient 10 can include age, weight, sex, physical condition (e.g. pregnant), and nature of injury. This information can be used to determine the correct critical value and, if necessary, the proper factors of safety to warn the caregiver prior to reaching the critical value.

Additional embodiments can include a Global Positioning System (GPS) 126, either separate from or included with the monitor 112. GPS 126 can provide positional and velocity information regarding the vehicle. The velocity information can be correlated with the sensor data 110 to determine the safe travel speed of the vehicle to keep the forces applied to the patient 10 below the critical value. Route information as well as the safe travel speed can be displayed on display 120.

The monitor 112 can also include a transmitter/receiver 128 linked to an external radio source or network (e.g. LAN, WAN or Internet) to receive traffic and road condition information. The traffic and road information can be accessed periodically or accessed in real time to allow for “on-the-fly” changes to the travel route of the vehicle. The traffic and road information can be accessed from a local traffic report, updates provided by other caregivers traveling to the same destination and transportation department databases listing the locations of road surface problems (e.g. open construction sites, road imperfections, raised surfaces, steel plates, and potholes).

As an example, the caregiver can input the destination and receive a potential route from his present location to the destination, e.g. the hospital. The route can be analyzed for fastest travel time, traffic conditions and potential road surface problems and can be changed accordingly. Alternately, or in addition, the system 100 can provide the suggested speed of the vehicle while traveling the route and monitor the speed of the vehicle for compliance.

In another embodiment, a camera 130 can be positioned to monitor the patient 10. The camera 130 can be a full-motion camera or a still-motion camera that takes images at preset intervals. The image data can be transmitted to the display 120 for the caregiver to monitor the patient 10 if they are in separate sections of the vehicle.

In another embodiment, the type of mattress 12 that the patent is resting on can be inputted via the input device 124. Different mattresses provide different protection to impulse forces (see the example below). The monitor 112 can take the difference in the impulse absorbent qualities of the mattresses into account when analyzing and outputting the data. Alternately, or in addition to the mattress 12, a supplemental support 132 can also be included. The supplemental support 132 can be an adjustable support, e.g. an air mattress, and can be monitored and controlled by monitor 112. For example, a pressure monitor and compressor 134 can be used to monitor the status of the supplemental support 132. As the sensor data 110 approaches the critical value, the supplemental support 132 can be adjusted to dampen the forces applied to the patient 10 to lower the sensor data 110 to an acceptable level below the critical value (e.g. the mattress can be inflated and deflated as necessary). Additionally, the supplemental support 132 can be used for emergency purposes and only deploy if the sensor data 110 exceeds the critical value or exceeds the critical value by a certain percentage.

Regarding all embodiments, the memory 118 can be used to store any or all of the information outlined above for future review.

The above embodiments are described in light of transporting the patient 10 between two facilities by vehicle. In addition, the present invention can also be used for intra-facility transportation between floors and rooms within the same facility.

Some examples of impulse forces for both inter-facility and intra-facility transportation are outlined below.

### EXAMPLE 1—INTER-FACILITY TRANSPORT

Five roundtrip trials were conducted using a common route, having a mean distance of 4 miles, for transferring a neonate between facilities. Transportation was provided using a standard medical ambulance and transport isolette. During the trials, 5 acceleration measurements were made per second in the X (front-to-back), Y (side-to-side), and Z (up-and-down) planes using a computerized accelerometer attached to a neonatal resuscitation mannequin. The mannequin was first placed in the standard isolette without a mattress and the second trial was performed with the isolette outfitted with an air-foam mattress. The baseline values were obtained from placing the sensor on the mannequin and leaving the mannequin in an isolette at rest. This provided the baseline readings from the sensor to calibrate the sensor.

The results were mathematically integrated over the trial time in each dimension to yield a measure of impulse (force-per-unit-time). Total impulse for the trial was calculated using vector summation. A total of 281,457 data points were analyzed and the results are summarized below:

TABLE 1

| | | | | | | P Value |
|---|---|---|---|---|---|---|
| | Mean Time | Mean Impulse ± SD | | | | Compared |
| | per Trial | X | Y | Z | (m/sec²/min) | with |
| | (min.) + SD | (Front to Back) | (Side to Side) | (Up and Down) | Total | Baseline |
| Baseline | 11.10 ± 0.038 | 0.395 ± 0.318 | 0.392 ± 0.479 | 0.382 ± 0.136# | 1.02 ± 0.593 | |
| Standard | 10.91 ± 3.59 | 3.76 ± 2.34* | 3.13 ± 3.47 | 1.00 ± 1.06* | 28.82 ± 4.46 | ≦0.05 |
| Isolette | | | | | | |
| Standard | 9.32 ± 1.23 | 2.20 ± 1.82* | 2.68 ± 2.46 | 0.397 ± 0.30*# | 26.87 ± 2.34 | ≦0.05 |
| Isolette + | | | | | | |
| Airfoam | | | | | | |
| Mattress | | | | | | |

Probability Value:
*= P ≦ 0.05 - Denotes Statistical Significance
#= P ≧ 0.05 - Denote lack of Statistical Significance

As can be seen, neonates transported with the air-foam mattress plus isolette experienced less impulse in the X (front-to-back) and Z (up-and-down) dimensions as compared to a standard isolette without a mattress.

### EXAMPLE 2—INTRA-FACILITY TRANSPORT

Five transport trials were conducted from a delivery room to a neonatal intensive care unit (NICU) utilizing four different transport configurations with a standard neonatal isolette outfitted with a gel pillow, an air-foam mattress, and the air-foam mattress with the gel pillow. The results were mathematically integrated over the trial time in each dimension to yield a measure of impulse (force-per-unit-time). Total impulse for the trial was calculated using vector summation. A total of 60,756 data points were analyzed and the results are summarized below:

TABLE 2

| | | | | | | P Value |
|---|---|---|---|---|---|---|
| | Mean Time | Mean Impulse ± SD | | | | Compared |
| | per Trial | X | Y | Z | (m/sec²/min) | with |
| | (min.) + SD | (Front to Back) | (Side to Side) | (Up and Down) | Total | Standard |
| Standard | 5.59 ± 1.10 | 0.27 ± 0.24 | 0.22 ± 0.21 | 0.16 ± 0.64 | 5.82 ± 0.13 | |
| Isolette | | | | | | |
| Standard | 4.97 ± 1.64 | 0.74 ± 0.56 | 0.22 ± 0.27 | 0.06 ± 0.06 | 4.84 ± 0.16* | ≦0.05 |
| Isolette + | | | | | | |
| Gel | | | | | | |
| Pillow | | | | | | |
| Airfoam | 4.91 ± 1.12 | 0.20 ± 0.20 | 0.25 ± 0.17 | 0.36 ± 0.43 | 4.26 ± 0.22* | ≦0.05 |
| Mattress | | | | | | |
| Airfoam | 4.788 ± 0.92 | 0.45 ± 0.41 | 0.16 ± 0.13 | 0.18 ± 0.13 | 4.18 ± 0.36* | ≦0.05 |
| Mattress + | | | | | | |
| Gel | | | | | | |
| Pillow | | | | | | |

Probability Value:
*= P ≦ 0.05 - Denotes Statistical Significance
# = P ≧ 0.05 - Denote lack of Statistical Significance

As can be seen, neonates transported using the air-foam mattress and gel pillow experienced significantly less total impulse.

The above values can be used to correlate the tolerance and critical values for transporting the patient 10. Values for neonate sensitivity to transportation can be considered the most sensitive data and used as a base line for all patients, including infants and adult trauma patients. Alternately, testing can be performed over a range of patient conditions, ages and sexes to determine separate baselines for each grouping.

While there have been shown, described, and pointed out fundamental novel features of the invention as applied to a preferred embodiment thereof, it will be understood that various omissions, substitutions, and changes in the form and details of the devices illustrated, and in their operation, may be made by those skilled in the art without departing from the spirit and scope of the invention. For example, it is expressly intended that all combinations of those elements and/or steps which perform substantially the same function, in substantially the same way, to achieve the same results are within the scope of the invention. Substitutions of elements from one described embodiment to another are also fully intended and contemplated. It is also to be understood that the drawings are not necessarily drawn to scale, but that they are merely conceptual in nature.

1. A device to detect and monitor forces applied to a patient during transport, comprising:
a sensor attached to the patient generating sensor data; and
a monitor linked to the sensor and receiving the sensor data and comprising:
a memory storing a critical value;
a processor comparing the sensor data to the critical value; and
an alarm annunciating a critical condition;
wherein if the sensor data is greater than or equal to the critical value, the critical condition is determined and the alarm is activated.

2. An apparatus for monitoring acceleration of a living being during movement of the living being, the apparatus comprising:
a sensor positioned adjacent the living being, the sensor operative to detect acceleration of the living being at predetermined times while the living being is moving, the sensor further being operative to provide signals corresponding to the detected acceleration;
a monitor operative to receive the sensor signals, the monitor comprising:
a memory for storing sensor data corresponding to the sensor signals; and
a processor unit operative to analyze the sensor data and generate an alarm signal when the sensor data indicates that the acceleration of the living being exceeds a predetermined threshold.

3. The apparatus of claim 2 wherein the sensor is positioned adjacent a skull of the living being.

4. The apparatus of claim 3, further comprising a band, hat, skullcap or adhesive for positioning the sensor.

5. The apparatus of claim 2, wherein the sensor comprises a one-axis accelerometer.

6. The apparatus of claim 2, wherein the sensor comprises a three-axis accelerometer.

7. The apparatus of claim 2, further comprising a wireless transmitter in communication with the sensor for transmitting the sensor signals to the monitor.

8. The apparatus of claim 2, wherein the processor unit is operative to analyze the sensor data substantially in real time.

9. The apparatus of claim 8, wherein the memory is operative to store the analyzed sensor data.

10. The apparatus of claim 2, further comprising a display for displaying information corresponding to the sensor data.

11. The apparatus of claim 2, wherein the processor unit is operative to determine a suggested speed for transporting said living being such that the acceleration of the living being when the living being is transported at a speed below said suggested speed maintains the acceleration below the predetermined threshold.

12. The apparatus of claim 2, wherein the alarm signal includes an audio signal.

13. The apparatus of claim 2, wherein the alarm signal includes a visual signal.

14. The apparatus of claim 2, further comprising a user input device for receiving operational information from a user.

15. The apparatus of claim 14, wherein the operational information includes information specific to the living being and which is used to determine the predetermined threshold.

16. The apparatus of claim 2, further comprising a GPS unit.

17. The apparatus of claim 16, wherein the monitor is operative to receive traffic information or road information and the processor unit and GPS unit are operative to determine a route of travel based on the traffic information or the road information.

18. The apparatus of claim 2, further comprising a camera for providing an image of the living being.

19. The apparatus of claim 2, further comprising an adjustable support for the living being, wherein said support is adjusted based on the detected acceleration.

20. The apparatus of claim 2, wherein the processor unit is operative to determine a time duration for the detected acceleration and to determine an impulse applied to the living being, the impulse being based on the detected acceleration and time duration.

21. An apparatus for monitoring acceleration of a living being during movement of the living being in a vehicle, the apparatus comprising:
a three-axis accelerometer sensor positioned adjacent a skull of the living being, the sensor operative to detect acceleration of the living being in each of three mutually orthogonal axes at predetermined times while the living being is in the vehicle as the vehicle travels along a road, the sensor further being operative to provide signals corresponding to the detected acceleration;
a monitor operative to receive the sensor signals, the monitor comprising:
a memory for storing sensor data corresponding to the sensor signals;
a user input device for allowing a user to input information related to the living being;
a processor unit operative to analyze the sensor data taking into account the user input information and generate an alarm signal when the sensor data indicates that the acceleration of the living being exceeds a predetermined threshold;
a display for displaying information corresponding to the sensor data, the displayed information representing impulse conditions experienced by the living being as a result of the detected acceleration;
the processor unit further being programmed to perform the following steps:
receive information regarding a physical condition of a first roadway;
receive information regarding a physical condition of a second roadway;
select one of the first roadway and the second roadway based on the physical condition of the roadways such that the selected roadway will result in reduced acceleration experienced by the living being as the vehicle travels along the selected roadway in comparison with the acceleration experienced along the roadway which is not selected.

22. A method for monitoring acceleration of a living being during movement of the living being, the method comprising the following steps:
detecting acceleration of the living being at predetermined times while the living being is moving using a sensor positioned adjacent the living being, the sensor operative to provide signals corresponding to the detected acceleration;
receiving the sensor signals at a monitor;
storing sensor data in a memory associated with the monitor, the sensor data corresponding to the sensor signals; and
analyzing, using a processor unit, the sensor data and generating an alarm signal when the sensor data indicates that the acceleration of the living being exceeds a predetermined threshold.

23. The method of claim 22 further comprising the step of positioning the sensor adjacent a skull of the living being.

24. The method of claim 23, further comprising the step of using a band, hat, skullcap or adhesive for positioning the sensor.

25. The method of claim 22, wherein the sensor comprises a one-axis accelerometer.

26. The method of claim 22, wherein the sensor comprises a three-axis accelerometer.

27. The method of claim 22, further comprising the step of transmitting the sensor signals to a monitor using a wireless transmitter in communication with the sensor.

28. The method of claim 22, further comprising the step of analyzing the sensor data substantially in real time using a processor unit.

29. The method of claim 28, further comprising the step of storing the analyzed sensor data in a memory.

30. The method of claim 22, further comprising the step of displaying information corresponding to the sensor data.

31. The method of claim 22, further comprising the step of determining a suggested speed for transporting said living being such that the acceleration of the living being when the living being is transported at a speed below said suggested speed maintains the acceleration below the predetermined threshold.

32. The method of claim 22, wherein the alarm signal includes an audio signal.

33. The method of claim 22, wherein the alarm signal includes a visual signal.

34. The method of claim 22, further comprising the step of receiving operational information from a user by way of an input device.

35. The method of claim 34, wherein the operational information includes information specific to the living being and which is used to determine the predetermined threshold.

36. The method of claim 22, further comprising the step of using a GPS unit.

37. The method of claim 36, further comprising the steps of receiving traffic information or road information and using the GPS unit to determine a route of travel based on the traffic information or the road information.

38. The method of claim 22, further comprising the step of using a camera to provide an image of the living being.

39. The method of claim 22, further comprising the step of adjusting an adjustable support for the living being, wherein said support is adjusted based on the detected acceleration.

40. The method of claim 22, further comprising the steps of determining a time duration for the detected acceleration and determining an impulse applied to the living being, the impulse being based on the detected acceleration and time duration.

41. A method for monitoring acceleration of a living being during movement of the living being in a vehicle, the method comprising the following steps:
detecting acceleration of the living being in each of three mutually orthogonal axes at predetermined times using a three-axis accelerometer sensor positioned adjacent a skull of the living being while the living being is in the vehicle as the vehicle travels along a road, the sensor further being operative to provide signals corresponding to the detected acceleration;
storing sensor data corresponding to the sensor signals in a memory;
inputting information related to the living being;
analyzing the sensor data taking into account the user input information and generating an alarm signal when the sensor data indicates that the acceleration of the living being exceeds a predetermined threshold;
displaying information corresponding to the sensor data, the displayed information representing impulse conditions experienced by the living being as a result of the detected acceleration;
receiving information regarding a physical condition of a first roadway;
receiving information regarding a physical condition of a second roadway;
selecting one of the first roadway and the second roadway based on the physical condition of the roadways such that the selected roadway will result in reduced acceleration experienced by the living being as the vehicle travels along the selected roadway in comparison with the acceleration experienced along the roadway which is not selected.
